(19) United States
(12) Patent Application Publication
(10) Pub. No.: US 2019/0076112 A1
(43) Pub. Date: Mar. 14, 2019

(54) Ultrasound-Enabled Fiducials for Real-Time Detection of Brain Shift During Neurosurgery

(71) Applicant: Greenville Neuromodulation Center, Greenville, PA (US)

(72) Inventor: Erwin B. Montgomery, JR., Dundas (CA)

(21) Appl. No.: 16/130,196

(22) Filed: Sep. 13, 2018

Related U.S. Application Data
(60) Provisional application No. 62/558,644, filed on Sep. 14, 2017.

(51) Int. Cl. A61B 8/08; A61B 90/00

(52) U.S. Cl. CPC A61B 8/0808; A61B 90/39; A61B 90/11; A61B 8/485; A61B 90/36

(57) ABSTRACT

Provided herein are devices, systems, and methods, for capturing perioperative intracranial ultrasound images and for determining elasticity of brain tissue.

### CROSS-REFERENCE TO RELATED APPLICATION

The present application claims the benefit of U.S. Provisional Patent Application No. 62/558,644, filed Sep. 14, 2017, the contents of which are incorporated herein by reference in their entirety.

### BACKGROUND OF THE INVENTION

### Field of the Invention

The present invention is directed to devices, systems, and methods for neurosurgery.

In particular, the present invention is directed to fiducials enabled for use with ultrasound during neurosurgery.

### Description of Related Art

Typically, in preparing for surgical interventions involving a patient's central nervous system (CNS), detailed planning is involved. This planning includes use of imaging technologies in a pre-surgical planning phase, for example, magnetic resonance imaging (MRI) or computed tomography (CT or CAT) scanning, to allow the surgical team to identify targets and visualize critical CNS structures to be avoided during the surgical intervention.

To provide a reference point in such images, in particular, for surgical interventions that make use of stereotactic frames for positioning and guidance during surgery, fiducials or fiducial markers are typically placed in or on the patient during imaging. Such fiducials typically take the form of bone anchors implanted in the skull, or stickers including a radiopaque component, such that the fiducials are visible in the obtained image.

However, there are issues with regard to the use of standard imaging techniques in the pre-surgical phase, particularly with regard to neurosurgery. A significant concern is the phenomenon known as “brain shift.” See Winkler et al., “The first evaluation of brain shift during functional neurosurgery by deformation field analysis.” J. Neurol. Neurosurg. Psychiatry 2005, 76: 1161-1163. Shifting of the brain, for example, due to placing the patient in a particular position for a prolonged period of time, or differences in patient positioning between pre-surgical imaging and the surgical intervention, can result in a loss of positional accuracy. A loss of accuracy can result in the surgical path passing through critical anatomical structures, the intervention being unsuccessful in terms of reaching a desired anatomical target, or both.

Similarly, limitations in previous devices and methods prevented the use of standard imaging techniques for assessing intracranial blood flow and for measuring tissue elasticity in the brain perioperatively.

In view of these shortcomings, brain shift, changes in tissue elasticity, and changes in blood flow heretofore have gone unrecognized until after the procedure, when interventions may not be possible or may have reduced effectiveness. Recent advances in stereotactic technology have allowed image acquisition during the surgical procedure. See Ivan et al., “Brain shift during bur hole-based procedures using interventional MRI.” J. Neurosurg 2014, 121: 149-160. However, the use of traditional imaging techniques such as MRI and CT during the surgical procedure is expensive and time-consuming.

Therefore, a need exists in the art to allow for intraoperative imaging to recognize and account for shifting of internal anatomy and changes in internal physiology.

### SUMMARY OF THE INVENTION

To address this need, the invention described herein makes use of ultrasound imaging. Ultrasound imagining is an inexpensive, rapid means of obtaining images of internal anatomy. However, with regard to neurosurgery, in particular, ultrasound is not possible, due to the thickness and density of the skull, in particular, the outer table of the skull.

Accordingly, provided here are fiducials having ultrasound transducers coupled thereto. In aspects, the fiducials comprise bone anchors that are implanted into a patient's skull. The bone anchors include a hollow shaft into which an ultrasound transducer is coupled, either permanently or reversibly. The bone anchor is implanted within the skull at a level, such that, the outer table is traversed, thus, the ultrasound transducer need only penetrate the thin inner table to acquire images of internal anatomy.

Also provided herein is a system including an ultrasound-enabled fiducial having a proximal end configured for attachment to a patient and to bypass an outer table of the patient's skull; a distal end; and an ultrasound transducer; and one or more processors in communication with the transducer, the one or more processors programmed or configured to transmit, by a signal generator, a first electrical signal to the transducer; receive first ultrasound data from the transducer; and convert the first ultrasound data to a first ultrasound image.

Also provided herein is a method of obtaining an intracranial ultrasound image, including the steps of attaching to a patient's skull an ultrasound-enabled fiducial having a proximal end configured for attachment to a patient and to bypass an outer table of the patient's skull; a distal end; and an ultrasound transducer; and generating an ultrasound image using that fiducial. In aspects, the ultrasound image is a traditional structural ultrasound, a B-mode ultrasound, a Doppler ultrasound, or an ultrasound elastograph.

Also provided herein is a method of detecting changes in elasticity of a patient's brain tissue, including the step of attaching to a patient's skull an ultrasound-enabled fiducial having a proximal end configured for attachment to a patient and to bypass an outer table of the patient's skull; a distal end; and an ultrasound transducer. The method further includes the steps of providing an electrical signal to the transducer; converting, with the transducer, the electrical signal to sound waves; receiving, with the transducer, reflected sound waves; converting, with the transducer, the reflected sound waves to first ultrasound data; receiving, with one or more processors and from the transducer, the first ultrasound data converting, with one or more processors, the first ultrasound data to first elasticity data; providing an electrical signal to the transducer; converting, with the transducer, the electrical signal to sound waves; receiving, with the transducer, reflected sound waves; converting, with the transducer, the reflected sound waves to second ultrasound data; receiving, with one or more processors and from the transducer, the second ultrasound data; converting, with one or more processors, the second ultrasound data to second elasticity data; calculating, with one or more processors, a difference in elasticity in the brain tissue based on the first elasticity data and the second elasticity data; determining, with one or more processors and based on a predetermined threshold, whether the difference in elasticity exceeds the predetermined threshold; and providing a visual, audible, and/or tactile alert if the difference exceeds the predetermined threshold.

Further aspects are set forth in the following numbered clauses:

Clause 1: An ultrasound-enabled fiducial comprising: a proximal end configured for attachment to a patient; a distal end; and an ultrasound transducer.

Clause 2: The ultrasound-enabled fiducial of clause 1, wherein the proximal end comprises a screw region.

Clause 3: The ultrasound-enabled fiducial of clause 2, wherein the fiducial is self-tapping.

Clause 4: The ultrasound-enabled fiducial of clause 2, wherein the fiducial is self-drilling.

Clause 5: The ultrasound-enabled fiducial of clause 2, wherein the fiducial is self-tapping and self-drilling.

Clause 6: The ultrasound-enabled fiducial of any of clauses 1-5, wherein the fiducial is radiopaque and, optionally is MRI-compatible.

Clause 7: The ultrasound-enabled fiducial of any of clauses 1-6, wherein the fiducial is made from a metal.

Clause 8: The ultrasound-enabled fiducial of any of clauses 1-7, wherein the fiducial is made from one or more of titanium, titanium alloys, cobalt chrome, and/or stainless steel.

Clause 9: The ultrasound-enabled fiducial of clause 1, wherein the fiducial is made of a polymeric material.

Clause 10: The ultrasound-enabled fiducial of any of clauses 1-9, wherein the ultrasound transducer comprises an ultrasound receiver and an ultrasound transmitter.

Clause 11: The ultrasound-enabled fiducial of any of clauses 1-10, wherein the fiducial comprises a hollow shaft between the proximal end and the distal end.

Clause 12: The ultrasound-enabled fiducial of clause 11, wherein the hollow shaft is at least partially filled with an ultrasound gel.

Clause 13: A system comprising an ultrasound-enabled fiducial comprising a proximal end configured for attachment to a patient; a distal end; and an ultrasound transducer; and one or more processors in communication with the transducer, the one or more processors programmed or configured to transmit, by a signal generator, a first electrical signal to the transducer; receive first ultrasound data from the transducer; and convert the first ultrasound data to a first ultrasound image.

Clause 14: The system of clause 13, wherein one or more processors are further programmed or configured to store, in a memory, the first ultrasound data and/or the first ultrasound image; transmit, by a signal generator, a second electrical signal to the transducer; receive second ultrasound data from the transducer; and convert the second ultrasound data to a second ultrasound image.

Clause 15: The system of clause 14, wherein one or more processors are further programmed or configured to identify, based on the first ultrasound data and/or the first ultrasound image, one or more anatomical landmarks; identify, in the second ultrasound data and/or second ultrasound image, the one or more anatomical landmarks; and calculate, between the first ultrasound data and/or image and the second ultrasound data and/or image, a difference in location of the one or more anatomical landmarks.

Clause 16: The system of clause 15, wherein one or more processors are further programmed or configured to in response to calculating the difference in location and based on a predetermined threshold, provide a visual, audible, and/or tactile alert if the difference exceeds the predetermined threshold.

Clause 17: A method of obtaining an intracranial ultrasound image, comprising attaching to a patient's skull one or more ultrasound-enabled fiducials, the one or more fiducials comprising a proximal end configured for attachment to the patient's skull; a distal end; and an ultrasound transducer; and generating an ultrasound image.

Clause 18: The method of clause 17, wherein the step of generating an image comprises providing an electrical signal to the transducer; converting, with the transducer, the electrical signal to sound waves; receiving, with the transducer, return sound waves; converting, with the transducer, the return sound waves to ultrasound data; receiving, with one or more processors and from the transducer, the ultrasound data; and converting, with one or more processors, the ultrasound data to an ultrasound image.

Clause 19: The method of clause 18, wherein the ultrasound data is Doppler ultrasound data and the ultrasound image is a Doppler ultrasound image, and wherein the method further comprises the step of introducing a contrast agent into the patient's circulation.

Clause 20: The method of clause 17 or clause 18, wherein in the ultrasound-enabled fiducial comprises a bone anchor, and wherein the bone anchor is inserted into the patient's skull such that the ultrasound transducer is located interiorly of the outer table of the patient's skull, or can transmit soundwaves through the fiducial that bypass the outer table.

Clause 21: The method of any of clauses 17, 18, or 20, wherein the method further comprises the steps of identifying, with one or more processors, one or more anatomical landmarks in a first ultrasound image; identifying, with one or more processors, one or more anatomical landmarks in a second ultrasound image; and calculating, between the first ultrasound image and the second ultrasound image, a difference in location of the one or more anatomical landmarks.

Clause 22: The method of clause 21, further comprising the step of in response to calculating the difference in location and based on a predetermined threshold, providing a visual, audible, and/or tactile alert if the difference exceeds the predetermined threshold.

Clause 23: A method of calculating tissue elasticity in a patient's brain comprising the steps of attaching to a patient's skull one or more ultrasound-enabled fiducials, the one or more fiducials comprising a proximal end configured for attachment to the patient; a distal end; and an ultrasound transducer, wherein the proximal end of the fiducial bypasses an outer table of the patient's skull; transmitting, by a signal generator, a first electrical signal to the transducer; receiving, with one or more processors, first ultrasound data from the transducer; and converting, with one or more processors, the first ultrasound data to first elasticity data.

Clause 24: The method of clause 23, further comprising converting, with one or more processors, the first elasticity data to a first elasticity image.

Clause 25: The method of clause 24, further comprising overlaying the first elasticity image on a CT image or an MRI image of the patient's brain.

Clause 26: The method of any of clauses 23-25, further comprising storing, in a memory, the first elasticity data; transmitting, by a signal generator, a second electrical signal to the transducer; receiving second ultrasound data from the transducer; and converting the second ultrasound data to second elasticity data.

Clause 27: The method of clause 26, further comprising converting, with one or more processors, the second elasticity data to a second elasticity image.

Clause 28: The method of clause 27, further comprising overlaying the second elasticity image on a CT image or an MRI image of the patient's brain.

Clause 29: The method of any of clauses 26-28, further comprising calculating, with one or more processors, a difference in elasticity in the brain tissue based on the first elasticity data and the second elasticity data, determining, with one or more processors and based on a predetermined threshold, whether the difference in elasticity exceeds the predetermined threshold, and, providing a visual, audible, and/or tactile alert if the difference exceeds the predetermined threshold.

### BRIEF DESCRIPTION OF THE DRAWINGS

FIG. 1 shows a bone anchor with an ultrasound transducer included therein according to one aspect of the present invention.

FIG. 2 shows a theoretical B-mode ultrasound according to one aspect of the present invention.

FIG. 3 shows a schematic representation of a system according to one aspect of the present invention.

### DESCRIPTION OF THE INVENTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

All references cited within this specification are incorporated by reference herein in their entirety.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention can assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “patient” refers to members of the animal kingdom including, but not limited to, mammals and human beings and is not limited to humans or animals in a doctor-patient or veterinarian-patient relationship. “A patient” refers to one or more patients such that a treatment effective in “a patient” refers to a treatment shown effective in one patient or a statistically significant number of patients in a population of patients.

As used herein, the terms “communication” and “communicate” refer to the receipt, transmission, or transfer of one or more signals, messages, commands, or other type of data. For one unit or device to be in communication with another unit or device, means that the one unit or device is able to receive data from and/or transmit data to the other unit or device. A communication can use a direct or indirect connection, and can be wired and/or wireless in nature. Additionally, two units or devices can be in communication with each other even though the data transmitted can be modified, processed, routed, etc., between the first and second unit or device. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible. Any known electronic communication protocols and/or algorithms can be used such as, for example, TCP/IP (including HTTP and other protocols), WLAN (including 802.11 and other radio frequency-based protocols and methods), analog transmissions, Global System for Mobile Communications (GSM), and/or the like.

The figures accompanying this application are representative in nature, and should not be construed as implying any particular scale or directionality, unless otherwise indicated.

Provided herein are ultrasound-enabled fiducials. As used herein, the term “fiducial” means a fiducial marker suitable for providing a reference point in an image, for example, and without limitation, a pre- or perioperative image such as an MRI or CT-generated image. The fiducials can be bone anchors or any suitable structure that can be implanted within the skull to allow an ultrasound transducer to bypass the outer table, such that the ultrasound need only traverse the thin inner table of the skull. As used herein, the term “outer table” of the skull refers to the outermost, thick condensed layer of cranial bone. As used herein, the term “inner table” of the skull refers to the innermost, thin condensed layer of cranial bone. The outer table is separated from the inner table by cancellous bony tissue referred to as dipole.

In aspects, the fiducial is a bone anchor having a proximal end, a distal end, and a region therebetween connecting the proximal end and the distal end. The proximal end includes a base adapted or configured to be attached to a patient's body. In aspects, the base is adapted or configured to be attached to a bone, for example, and without limitation, the skull, in the patient's body. In aspects, the base includes a screw region to allow for implantation within the skull. In aspects, the screw region is self-tapping. In aspects, the screw region is self-drilling. In some aspects, the screw region is both self-tapping and self-drilling. In aspects, the anchor can be implanted without the need to pre-drill or pre-tap.

In aspects, the fiducial includes, at the distal end, a connector for allowing reversible coupling to a fiducial marker. In other aspects, the fiducial itself serves as the fiducial marker.

The fiducial can have any suitable length and width (circumference) and, in particular, the length and arrangement of the screw region can also be any suitable length, width, and configuration to allow for secure placement at a suitable depth to penetrate the outer table of the skull. Those of skill in the art will appreciate that table thickness is dependent on anatomical location. See Lillie et al., “Estimation of skull table thickness with clinical CT and validation with microCT.” J. Anat. 2015, 226(1): 73-80. For example, in human beings, the outer table overlaying the occipital lobe is significantly thinner than the outer table overlaying the frontal lobe. Thus, the fiducials described herein can vary in length to account for varying outer table thickness in a patient. In aspects, the fiducial is of sufficient length such that both the outer table and at least a portion of the dipole are traversed. In some aspects, the entire dipole, in addition to the outer table, is traversed. In some aspects, the fiducial is of sufficient length such that only the outer table is traversed.

In aspects, the fiducial is from 3 to 10 mm in length, all subranges therebetween inclusive. In aspects, the fiducial is about 5 mm in length, ±up to 0.5 mm. In aspects, the screw region is from 1 to 4 mm in length, all subranges therebetween inclusive. An example of a suitable set of parameters for a fiducial is found in the Waypoint™ Fiducial Anchor, commercially available from FHC, Inc. (Bowdoin, Me.).

The fiducial can be formed of any suitable material. In aspects, the fiducial is formed of any suitable, medical grade, biocompatible material. In aspects, particularly in aspects where the fiducial itself serves as a fiducial marker, the material is radiopaque, such that it is visible in an X-ray or CT-obtained image, or is MR-visible such that it can be seen in an MRI (e.g., it can also be MRI-compatible). In aspects, the fiducial is at least partially formed from a metal, such as titanium, titanium alloys, cobalt chrome, and/or stainless steel. In other aspects, the fiducial is at least partially polymeric and can be visualized with devices containing MRI-contrast to allow visualization by MRI, or is radiopaque and can be visualized in a CT scan. In aspects, the fiducial is formed of a material that generates a clear echogenic representation on an image.

The fiducial is configured such that an ultrasound (ultrasonic) transducer can be operably coupled therewith. In aspects, the ultrasound transducer is sized and configured such that both transmitting and receiving components of the transducer are coupled to the fiducial. In other aspects, the ultrasound receiver is inserted through a burr hole and the transmitting component is coupled to the fiducial, or vice versa. Miniaturized ultrasound transducers and/or probes are known to those of skill in the art (see, e.g., U.S. Pat. No. 4,977,898), and those of skill in the art will be able to select suitable miniaturized ultrasound transducers/probes (for example, and without limitation, as disclosed in U.S. Pat. No. 7,037,270, incorporated herein by reference in its entirety and Qiu et al., “Piezoelectric micromachined ultrasound transducer (PMUT) arrays for integrated sensing actuation and imaging” Sensors 2015, Vol. 15, pp. 8020-8041) to be utilized in the enabled fiducials described herein. Useful transducers/probes include those that are utilized in ultrasound catheters, for example, those available commercially from Phillips (Visions Digital Catheter) and Boston Scientific (OPTICROSS), as such transducers/probes are of sufficient size to be able to fit within the shaft of a fiducial as described herein.

In aspects, the ultrasound transducer is reversibly coupled to the fiducial, such that it can be attached and removed therefrom. This will typically occur where the ultrasound transducer is not MRI or CT-compatible. In aspects, the transducer is removable, and a plug or other temporary structure is inserted into the fiducial. In aspects, the ultrasound transducer is irreversibly coupled to the fiducial. In aspects, the irreversibly-coupled transducer is one that is MRI or CT-compatible. See Zaaroor et al., “Magnetic resonance-guided focused ultrasound thalamotomy for tremor: a report of 30 Parkinson's disease and essential tremor cases.” J. Neurosurg. 2017, 24:1-9. The fiducial and transducer are configured such that sound waves produced by the transducer bypass the outer table of the skull, and thus need only traverse the thinner inner table.

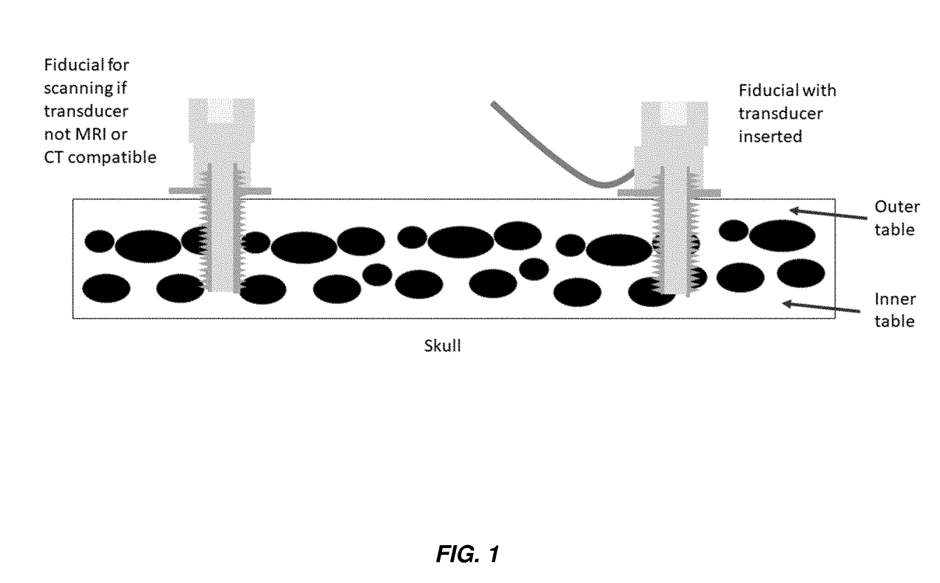

Turning to FIG. 1, in aspects the fiducial includes a partially or fully hollow shaft into which an ultrasound transducer can be placed. As shown in the figure, the fiducial is of sufficient length such that the outer table of the skull is bypassed, and the sound waves from the transducer need only traverse the thin inner table, allowing for ultrasound mapping of the brain. In aspects, the region between the proximal and distal ends of the fiducial is at least a partially hollow shaft, such that the ultrasound transducer can be inserted therein. In aspects, an ultrasound gel is included in the at least partially hollow shaft of the fiducial, and the transducer is not inserted therein. In aspects, an ultrasound gel is included in the hollow shaft of the fiducial, and the transducer is inserted therein or thereon. For example, in aspects, the transducer is not provided within the hollow shaft of the fiducial, but is located on a distal portion of the fiducial, but, because of the ultrasound gel within the shaft, can transmit sound waves that bypass the thick outer table of the skull. Suitable ultrasound gels are available commercially from any number of sources, for example and without limitation, from Parker Labs (Fairfield, N.J.), Next Medical (Branchburg, N.J.), and Covidien (Dublin, Republic of Ireland).

The ultrasound transducer can be a wired transducer, in wired communication with one or more processors, or the transducer may be wireless, in wireless communication with one or more processors. In aspects where the transducer is wireless, communication with one or more processors can be through any known wireless or near-field communication technology, such as Wi-Fi, Bluetooth, Zigbee, and the like.

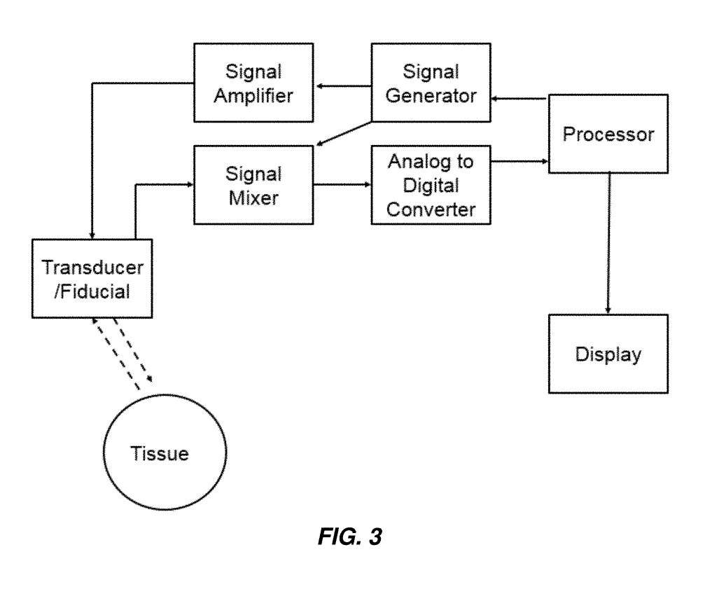

Also provided herein are systems including one or more fiducials as described herein and an ultrasound transducer coupled therewith. An exemplary, but non-limiting, system is illustrated schematically in FIG. 3, which shows a processor, which can control a signal generator to generate an electrical signal, which may be amplified, to excite a piezoelectric mechanism in a transducer to generate waves (e.g., sound waves, shown as the hatched line in the direction of the tissue), which is included in (removably or otherwise) an enabled fiducial as described previously. Signal generators, amplifiers, mixers, convertors, and software for controlling and analyzing data received from the same are known in the art and are available commercially, as separate components or whole systems, for example, from FujiFilm (VISUALSONICS), Phillips (EPIQ and AFFINITI), and General Electric Healthcare (LOGIQ and VIVID).

The transducer then receives the echoes (shown as the hatched line in the direction of the transducer) received from reflection of the waves at boundaries of various tissues/anatomical structures, wherein the echoes are converted and processed as is known in the art. In aspects, the system further includes one or more processors for causing an energy source to generate electrical signals that the transducer is configured to convert to sound waves, and for receiving electrical signals, including ultrasound data, that the transducer is configured to produce in response to the receipt of soundwaves. The one or more processors can be part of a computer including a display, a user input, and non-transitory memory, allowing for receipt of ultrasound data from the transducer, display of the ultrasound image, storage of the ultrasound data and/or ultrasound image, and processing/analysis of the ultrasound data and/or ultrasound image. In aspects, in which the transducer is a wireless transducer, the computer can be configured to receive such wireless signals and communicate the same to one or more processors.

In order to facilitate appropriate data communication and processing information between the various components of the computer, a system bus can be utilized. The system bus can be any of several types of bus structures, including a memory bus or memory controller, a peripheral bus, or a local bus using any of a variety of bus architectures. In particular, the system bus facilitates data and information communication between the various components (whether internal or external to the computer) through a variety of interfaces, as discussed hereinafter.

The computer can include a variety of discrete computer-readable media components. For example, this computer-readable media can include any media that can be accessed by the computer, such as volatile media, non-volatile media, removable media, non-removable media, etc. As a further example, this computer-readable media can include computer storage media, such as, media implemented in any method or technology for storage of information, such as, computer-readable instructions, data structures, program modules, or other data, random access memory (RAM), read only memory (ROM), electrically erasable programmable read only memory (EEPROM), flash memory, or other memory technology, CD-ROM, digital versatile disks (DVDs), or other optical disk storage, magnetic cassettes, magnetic tape, magnetic disk storage, or other magnetic storage devices, or any other medium which can be used to store the desired information and which can be accessed by the computer. Further, this computer-readable media can include communications media, such as computer-readable instructions, data structures, program modules, or other data in a modulated data signal, such as, a carrier wave or other transport mechanism and include any information delivery media, wired media (such as, a wired network and a direct-wired connection), and wireless media (such as, acoustic signals, radio frequency signals, optical signals, infrared signals, biometric signals, barcode signals, etc.). Of course, combinations of any of the above are included within the scope of computer-readable media.

The computer further includes a system memory with computer storage media in the form of volatile and non-volatile memory, such as ROM and RAM. A basic input/output system (BIOS), with appropriate computer-based routines, assists in transferring information between components within the computer and is normally stored in ROM. The RAM portion of the system memory typically contains data and program modules that are immediately accessible to or presently being operated on by a processing unit, e.g., an operating system, application programming interfaces, application programs, program modules, program data, and other instruction-based computer-readable codes.

The computer can also include other removable or non-removable, volatile or non-volatile computer storage media products. For example, the computer can include a non-removable memory interface that communicates with and controls a hard disk drive, i.e., a non-removable, non-volatile magnetic medium; and a removable, non-volatile memory interface that communicates with and controls a magnetic disk drive unit (which reads from and writes to a removable, non-volatile magnetic disk); an optical disk drive unit (which reads from and writes to a removable, non-volatile optical disk, such as a CD ROM); a Universal Serial Bus (USB) port for use in connection with a removable memory card, etc. However, it is envisioned that other removable or non-removable, volatile or non-volatile computer storage media can be used in the exemplary computing system environment including, but not limited to, magnetic tape cassettes, DVDs, digital video tape, solid state RAM, solid state ROM, etc. These various removable or non-removable, volatile or non-volatile magnetic media are in communication with the processing unit and other components of the computer via the system bus. The drives and their associated computer storage media discussed above provide storage of operating systems, computer-readable instructions, application programs, data structures, program modules, program data, and other instruction-based computer-readable code for the computer (whether duplicative or not of this information and data in the system memory).

A user can enter commands, information, and data into the computer through certain attachable or operable input devices, such as a keyboard, a mouse, etc., via a user input interface. Of course, a variety of such input devices can be utilized, e.g., a microphone, a trackball, a joystick, a touchpad, a touch-screen, a scanner, etc., including any arrangement that facilitates the input of data and information to the computer from an outside source. As discussed, these and other input devices are often connected to the processing unit through the user input interface coupled to the system bus but can be connected by other interface and bus structures, such as, a parallel port, game port, or a USB. Still further, data and information can be presented or provided to a user in an intelligible form or format through certain output devices, such as, a monitor (to visually display this information and data in electronic form), a printer (to physically display this information and data in print form), a speaker (to audibly present this information and data in audible form), etc. All of these devices are in communication with the computer through an output interface coupled to the system bus. It is envisioned that any such peripheral output devices can be used to provide information and data to the user.

The computer can operate in a network environment through the use of a communications device, which is integral to the computer or remote therefrom. This communications device is operable by and in communication with the other components of the computer through a communications interface. Using such an arrangement, the computer can connect with or otherwise communicate with one or more remote computers, such as a remote computer, which can be a personal computer, a server, a router, a network personal computer, a peer device, or other common network nodes, and typically includes many or all of the components described above in connection with the computer. Using appropriate communication devices, e.g., a modem, a network interface or adapter, etc., the computer can operate within and communicate through a local area network (LAN) and a wide area network (WAN), but can also include other networks such as a virtual private network (VPN), an office network, an enterprise network, an intranet, the Internet, etc. It will be appreciated that the network connections shown are exemplary and other means of establishing a communications link between the computers can be used.

With regard to a processor for generating electric signals, devices for producing suitable electrical signals for an ultrasound transducer are known to those of skill in the art and are commercially available from, for example and without limitation, GE Healthcare (Little Chalfont, United Kingdom), Phillips (Amsterdam, The Netherlands), and Siemens (Malvern, Pa.).

As used herein, the computer includes or is operable to execute appropriate custom-designed or conventional software to perform and implement the processing steps of the method and system of the present invention, thereby forming a specialized and particular computing system. Accordingly, the presently-invented method and system can include one or more computers or similar computing devices having a computer-readable storage medium capable of storing computer-readable program codes or instructions that causes the processing unit to execute, configure, or otherwise implement the methods, processes, and transformational data manipulations discussed hereinafter in connection with the present invention. Still further, the computer can be in the form of a personal computer, a personal digital assistant, a portable computer, a laptop, a palmtop, a mobile device, a mobile telephone, a server, or any other type of computing device having the necessary processing hardware to appropriately process data to effectively implement the presently-invented computer-implemented method and system.

Processing/analysis of the ultrasound data can include capture of discrete images from the ultrasound data, and comparison of images captured at various timepoints during the entire perioperative process, such that, changes including brain shift can be identified. Analysis of brain shift can be based on one or more anatomical landmarks that are included in the ultrasound data including, for example, and without limitation, one or more ventricles, one or more nuclei, or one or more white matter tracts.

Also provided herein are methods of assessing a change in state of a patient during surgery. In aspects, the surgery is neurosurgery. In aspects, the change in state is a shifting of an anatomical structure or landmark. In aspects, the anatomical structure is one or more portions of the central nervous system. In aspects, the anatomical structure is at least a portion of the brain of the patient. The method includes the steps of attaching one or more fiducial devices as described herein to a patient's skull. The fiducial devices are attached, or implanted, in such a manner that the thick outer table of the skull is at least partially bypassed. In aspects, the thick outer table is completely bypassed. In some aspects, the fiducial includes an ultrasound transducer permanently affixed thereto. In other aspects, the ultrasound transducer is removably coupled to the fiducial.

The method further includes the steps of acquiring, with the ultrasound transducer and one or more processors as described herein in communication therewith, ultrasound data and generating, with one or more processors, one or more ultrasound images, by generating high frequency (e.g., 20 KHz and above) waves, in some aspects sound waves, propagating the waves into the tissue of interest, and monitoring and collecting the reflected waves. As is known in the art, images of anatomical structures can then be generated based on the differences in density in various anatomical structures and the various rates of reflection of the waves at the boundaries between these anatomical structures. In aspects, the method further includes the step of identifying one or more anatomical structures and comparing, between images, location data relating to the one or more anatomical structures, such that shifting of the anatomical structure can be identified.

In aspects, anatomical shift, such as a brain shift, is continuously assessed by automatic and constantly-updating comparisons/analyses performed by one or more processors as new ultrasound data is received from the transducer. In aspects, one or more processors are programmed or configured to provide, automatically, an audible, visual, and/or tactile alert that a certain threshold of variance between images has been passed such that it is more likely than not that a shift has occurred in the anatomy.

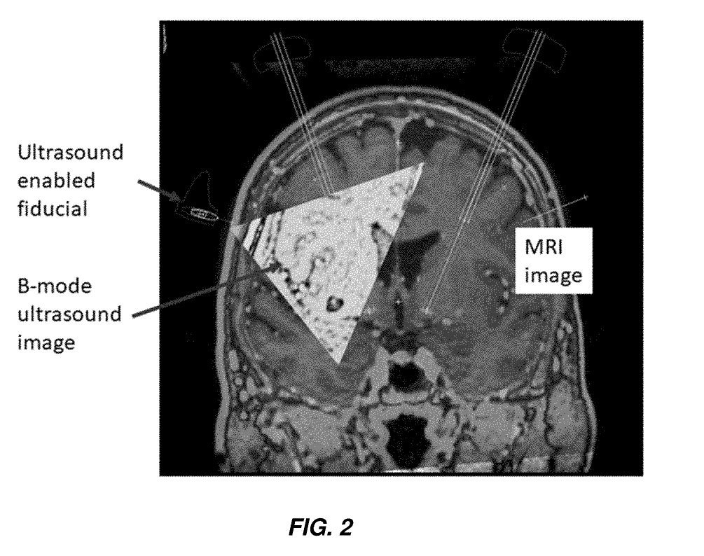

In aspects, the method includes obtaining a B-mode image (see. e.g., FIG. 2) with the ultrasound-enabled fiducials described herein. As used herein, B-mode ultrasound means a two-dimensional ultrasound, achievable with an array of transducers. With the present devices, systems, and methods, a plurality of ultrasound enabled fiducials can be implanted into a patient's skull for acquiring a B-mode ultrasound. This B-mode ultrasound image can, in aspects, be combined with an MRI or CT image, and/or with one or more additional images (for example, and without limitation, from other fiducials as described herein placed in other locations in a patient's skull) to generate a three-dimensional image. See, e.g., Pelizzari et al. “Accurate three-dimensional registration of CT, PET, and/or MR images of the brain.” J Comput Assist Tomogr. 1989, 13(1): 20-6; and Maintz et al. “Comparison of edge-based and ridge-based registration of CT and MR brain images.” Med Image Anal. 1996, 1(2): 151-61.

Thus, in other aspects, ultrasound images from multiple enabled fiducials can be co-registered with MRI or CT imaging used in image-based surgical navigation. This allows reformatting of the MRI and CT images to the fiducials' ultrasound view (similar to the probe's view in current ultrasound applications). Thus, the MRI and CT scans can be translated into an image of echogenicity that can be mapped with the ultrasound images using standard rotational and translational algorithms. See, e.g., Pelizzari et al. “Accurate three-dimensional registration of CT, PET, and/or MR images of the brain.” J Comput Assist Tomogr. 1989, 13(1): 20-6; and Maintz et al. “Comparison of edge-based and ridge-based registration of CT and MR brain images.” Med Image Anal. 1996, 1(2): 151-61. Any discrepancy between the MRI and CT images and the ultrasound images can alert the neurosurgeon, for example, by way of an audible, visual, and/or tactile alert automatically generated by one or more processors, to possible brain shift. Further, targeting can then be adjusted based on the ultrasound images.

The ultrasound enabled fiducials can be used to detect any change in neuroanatomy in a similar manner to how brain shift is detected, explained above. In some aspects, the fiducials can be used in methods of detecting intracranial events such as development of intracerebral hematomas and for providing up-to-the-second anatomy for biopsy procedures and/or ablation or resection procedures (including radiofrequency ablation procedures). In addition to a change in location of known anatomical structures due to shift in position of ultrasound signatures for known structures, the methods disclosed herein can detect a change in density brought on by, for example, a bleed in a particular area of the brain, which results in the “appearance” of new signatures due to a change in density of material. Thus, any condition that results in a change in density, such that an ultrasound signature would change (and not necessarily “move”), can be detected with the devices, systems, and methods disclosed herein.

In aspects, the ultrasound enabled fiducials can be used for targeting or locating medical devices during a wide range of procedures, such as, for example and without limitation, implantation of electrodes (other than DBS electrodes, which has already been discussed above), cannulae, catheters, devices for conducting ablation or inducing lesions (including by radiofrequency), intracranial encephalography, and neuroprosthetics.

In additional aspects, the ultrasound enabled fiducials can be used to assess changes in regional brain elasticity, including through ultrasound-based elastography (UE). The principles behind UE are known to those of skill in the art, see, e.g., Sigrist et al., “Ultrasound Elastography: Review of Techniques and Clinical Applications,” Theranostics 2017, Vol. 7, No. 5, pp. 1303-1329, and devices, systems, and methods utilizing the same are known from U.S. Pat. Nos. 6,099,471; 6,479,571; and 7,150,128, each incorporated by reference herein in their entirety. Accordingly, UE need not be described in full here. However, to date, intracranial applications of UE have been limited due to the difficulties associated with transmission through the outer table, which as described above possesses a thickness that impairs use of ultrasound technology. As known to those of skill in the art and as described more fully in the aforementioned review by Sigrist and colleagues, UE-based techniques, which relate to elasticity calculable by Hooke's Law (σ=Γ·ε, where stress (σ) is the force per unit area (in kilopascals, e.g., N/m²), ε is strain, and Γ is the elastic modulus, with higher values being indicative of a material that tends to resist deformation (has increased stiffness)), can be broadly classified into strain imaging techniques and shear wave imaging techniques.

Strain imaging techniques can be broadly classified as strain elastography and acoustic radiation force impulse. Strain elastography can be further subdivided based on the method of exciting the tissue. In a first aspect, a user can exert mechanical (ultrasound or other means) force on tissue with an ultrasound transducer. In a second aspect, an ultrasound transducer is maintained in a steady state, and tissue displacement is based on an internal physiologic action, such as respiration or the circulatory/cardiovascular system. Because cerebrospinal fluid is circulated through the central nervous system, displacement generated thereby may be suitable for use of strain elastography within the brain, similar to how circulation is useful for generating displacement for strain elastography in other anatomical areas, and can provide another basis for displacement aside from and/or in addition to the circulatory system. In strain imaging, the measure of modulus (Γ) is Young's modulus, which can be provided based on the following equation: σₙ=E·εₙ where σₙ is a normal stress that causes a normal strain (εₙ), and E is Young's modulus, where normal means perpendicular to the surface. Tissue displacement (measured in the same direction as the applied stress) can be measured by radiofrequency echo correlation-based tracking, Doppler processing, and combinations thereof.

In acoustic radiation force impulse imaging, displacement is provided by a short-duration, high intensity acoustic impulse. This displacement is provided normal to the surface, and thus Young's modulus can be calculated based on the equation provided above.

For both classifications of strain imaging, results can be visualized as an overlay on a B-mode image (defined above), or may be visualized independently.

Shear wave imaging measures displacement parallel to an applied normal stress (unlike strain imaging described above). Shear wave imaging can be subdivided into three classes, one-dimensional transient elastography, point shear wave elastography, and two-dimensional shear wave elastography. In one-dimensional elastography, a mechanical vibration is provided, and ultrasound is measured from the same device, thus allowing detection of shear waves and their propagation speed through the tissue of interest. Shear wave speed (cₛ) can be used to calculate the shear modulus using the equation cₛ=√G/ρ, where G is the shear modulus and ρ is tissue density. Shear modulus (G) can then be used to calculate Young's modulus using the equation E=2(ν+1)G, where ν is Poisson's ratio (combining equations discussed above, the conversion can be expanded as E=3G=3 ρcₛ²). Poisson's ratio is the ratio of the proportional decrease in a lateral measurement to the proportional increase in length in a sample that is stretched elastically. Poisson's ratio for brain tissue has been calculated to be between 0.4 and 0.499 (see Schiavone et al., “In vivo measurement of human brain elasticity using a light aspiration device,” Medical Image Analysis, 2009, Vol. 13, No. 4, pp. 673-678).

Point shear wave elastography involves displacing tissue in a normal direction in a single direction, not unlike acoustic radiation force impulse imaging (described above). However, tissue displacement is not measured in point shear wave elastography. Rather, the acoustic wave is transformed to a shear wave by absorption of the acoustic energy, and shear wave speed perpendicular to the plane of excitation (cₛ) is calculated. This shear wave speed measurement can be converted to Young's modulus as described above.

Two-dimensional shear wave elastography makes use of an acoustic radiation force (e.g., not a single focal point as in acoustic radiation force impulse imagining and point shear wave elastography). Multiple foci are stimulated, in rapid succession (faster than shear wave speed), creating a wave cone and allowing for measurement of wave speed (or Young's modulus, as described above) in real-time.

In some instances, brain elasticity can be related to fluid levels in the brain, in the intracellular and/or extracellular compartment(s). Numerous conditions are known to affect such fluid levels. For example, vasogenic edema can lead to extra-cellular fluid while intra-cellular fluid changes are associated with cytotoxic changes. These changes can be detected by ultrasound methods. Many pathologies, such as ischemia, infarct, tumor, infection, among others, can cause changes in intra- and extra-cellular fluid that can be detectable by ultrasound-based elastography as described above.

Because of the ability of the fiducials of the present invention to bypass the thick outer table, elastography can be applied to the brain, as described herein, opening up new avenues of characterizing the brain and effects of various conditions, disease, and/or disorders thereof.

For example, animal studies have demonstrated changes in brain elasticity in response to experimental stroke (Xu et al. “Evidence of changes in brain tissue stiffness after ischemic stroke derived from ultrasound-based elastography.” J Ultrasound Med. 2013, Vol. 32, No. 3, pp. 485-94). Xu and colleagues showed that it is possible to detect shear moduli differences in ipsilateral tissue following middle cerebral artery occlusion (MCA occlusion—a model for stroke), and that, 24 hours after stroke, the ipsilateral hemisphere in animals subjected to MCA occlusion exhibited lower shear modulus (lesser elasticity) than in control animals, which is consistent with results seen using magnetic resonance elastography. Without wishing to be bound by the theory, intracellular and extracellular changes due to the lack of blood flow and oxygen (e.g., blood flow variations, pressure variations, and/or edema formation) are expected to manifest rapidly, such that detection of ischemia can occur perioperatively.

One among many applications of the present devices, systems, and methods is real-time monitoring to detect ischemia prior to infarction during neurovascular procedures to avoid irreversible injury. The present devices, systems, and methods can, in aspects, detect a change in elasticity of the brain brought upon by ischemia, which reduces oxygen availability, causing a dysregulation of water transport. This dysregulation can increase water content in the intracellular compartment, extracellular compartment, and/or both, and thus changes the elasticity of the brain, which can be detected by the UE methods disclosed herein.

Accordingly, provided herein is a method of determining a change in ultrasound measures of tissue, in particular brain tissue, in a patient during a neurovascular procedure or any operation that could result in an ischemic event in the brain or brain shift. In aspects, the method includes the steps of acquiring one or more ultrasound measurements (via the devices, systems, and methods disclosed herein) prior to beginning an operation, optionally determining a mean and standard deviation of those preoperative measurements, and periodically during the perioperative time acquiring additional ultrasound measurements (again via the methods disclosed herein). One or more processors can be programmed or configured to compare the preoperative ultrasound measurement data with one or more perioperative measures of ultrasound, and can be programmed or configured to cause an audible, visual, and/or tactile alert if a difference in ultrasound measurements between preoperative and one or more of the perioperative measurements exceeds a predetermined threshold. In aspects, an ultrasound image (generated based on the above-described methods) is divided into pixels, or small segments of the image, and statistical analyses are applied to the same pixel of data collected prior to and during a surgical intervention. Thus, a change in an echo boundary, such as, but not limited to, the boundary between the cerebrospinal fluid in a ventricle or in the subarachnoid space and the substance of the brain, measurement of blood flow, measurement of echogenic contrast, and elasticity can be utilized to detect brain shift and/or a pending ischemic event.

In some aspects, a change in ultrasound measurements that is statistically significant (p≤0.05) is set as a threshold for determining whether the ultrasound measurements of tissue has changed, such that a warning can be triggered by the systems disclosed herein. In some aspects, a change in the ultrasound measurements that is greater than or equal to 1.96 times the standard deviation of a set of elasticity measurements captured prior to beginning a surgical intervention is set as a threshold for determining whether ultrasound measurements of tissue has changed, such that a warning can be triggered by the systems disclosed herein. Those of skill in the art will appreciate that a surgeon or other professional can set a particular threshold for alerting the medical staff of a change in ultrasound measure, which change is indicative of an ischemic event or other perioperative blood flow issue or brain shift. In some aspects, the ultrasound data is elasticity data and the ultrasound measurement is a measurement of elasticity of brain tissue. In some aspects, the ultrasound data is blood flow data and the ultrasound measurement is a measurement of intracranial blood flow.

In aspects, changes in elasticity are continuously assessed by automatic and constantly-updating comparisons/analyses performed by one or more processors as new elasticity data is received from the transducer. In aspects, one or more processors are programmed or configured to provide, automatically, an audible, visual, and/or tactile alert when a change in elasticity beyond a predetermined threshold (as described above) is detected.

In aspects, changes in blood flow are continuously assessed by automatic and constantly-updating comparisons/analyses performed by one or more processors as new blood flow data is received from the transducer. In aspects, one or more processors are programmed or configured to provide, automatically, an audible, visual, and/or tactile alert when a change in blood flow beyond a predetermined threshold (as described above) is detected.

Examples of neurovascular procedures for which the present devices, systems, and methods are useful include, but are not limited to, aneurysm repair, removal of vascular anomalies, arterial bypass, and endarterectomies. However, virtually any intracranial procedure is at risk of producing vascular compromise, which the present devices, systems, and methods can detect and/or predict. In addition, procedures that are remote from the brain, but that nonetheless may cause ischemia and/or infarction in the brain, can benefit from the presently-disclosed devices, systems, and methods.

Another significant problem area in which the present devices, systems, and methods are useful is in brain tumor resection and changes in the brain anatomy and spatial location as the tumor is removed. A significant disparity can result between the preoperative MRI or CT imaging used to guide tumor resection and thus, the preoperative imaging becomes increasingly less useful and potentially misleading. This is a particularly significant problem with tumor resections done with minimally invasive stereotactic methods such that the neurosurgeon is unable to visualize the brain to note significant brain shifts. Consequently, some neurosurgeons have resorted to costly and technically demanding intra-operative MRI and CT scans. Ultrasound studies during open craniotomy demonstrate changes in elasticity of brain tumor tissue (Chauvet et al. “In Vivo Measurement of Brain Tumor Elasticity Using Intraoperative Shear Wave Elastography. Ultraschall Med. 2016, Vol. 37, No. 6, pp. 584-590). The ultrasound enabled fiducials can monitor brain shift in real-time based on ultrasound-based elastography, and the results thereof can be used to modify and update the preoperative MRI or CT scan imaging at lower costs and technical requirements.

Ultrasound has long been used to assess blood and other fluid flows, for example, using Doppler processing measures or intravascular ultrasound contrast, such as microbubbles and liposomes (see, e.g., Huang et al., “Liposomes as ultrasound imaging contrast agents and as ultrasound-sensitive drug delivery agents.” Cell Mol Biol Lett. 2002, Vol. 7, No. 2, pp. 233-5; Sever et al. “Dynamic visualization of lymphatic channels and sentinel lymph nodes using intradermal microbubbles and contrast-enhanced ultrasound in a swine model and patients with breast cancer.” J Ultrasound Med. 2010, Vol. 29, No. 12, pp. 1699-704). Contrast enhanced ultrasound (CEUS) agents, such as microbubbles, are being used to evaluate blood flow in large vessels (see, e.g., Rafailidis et al. “Evolving clinical applications of contrast-enhanced ultrasound (CEUS) in the abdominal aorta.” Cardiovasc Diagn Ther. 2018, Vol. 8, pp. S118-S130). However, recent research suggests that CEUS also may be able to detect hemodynamic changes in the microcirculation as well (see, e.g., Khaing et al. “Contrast-enhanced ultrasound to visualize hemodynamic changes after rodent spinal cord injury.” J Neurosurg Spine. 2018, Vol. 14, pp. 1-8). By in essence, removing the outer table of the skull (through the use of ultrasound enabled fiducials), these techniques can now be used to monitor blood flow through major intra-cranial blood vessels and, importantly, tissue micro-perfusion in real-time. The devices, systems, and methods disclosed herein can alert the neurosurgeon of a pending complication with potentially severe consequences in time to take remedial action.

Accordingly, in some aspects, a method includes an additional step of introducing a contrast agent and performing ultrasound analysis, for example, Doppler processing analysis of blood flow. An exemplary method includes the steps of implanting one or more ultrasound enabled fiducials as disclosed herein in the skull of a patient and administering one or more contrast agents to the circulatory system of the patient. Contrast agents, and methods of using the same in Doppler ultrasound methods, are known to those of skill in the art, for example, as described in Ignee et al. “Ultrasound contrast agents” Endosc. Ultrasound 2016, Vol. 5, No. 6, pp. 355-362. Suitable agents include compositions including encapsulated (e.g., shells of silica, liposomes, proteins, surfactants, etc.) inert gases and gas micro/nanobubble agents. The circulation carries the contrast agent and, combined with the devices, systems, and methods disclosed herein, allows the monitoring of blood flow through intra-cranial blood vessels using Doppler ultrasound methods, which are known in the art. Doppler ultrasound of intracranial blood flows has heretofore been limited, as have other cranial ultrasound methods, by poor signal impaired by the thick outer table of the skull. The present ultrasound-enabled fiducials overcome these prior issues and allow Doppler ultrasound monitoring of central blood flow. In this way, brain shift or changes in blood flow can be detected, based on a change in the Doppler signal. Moreover, changes in the Doppler signal can be used to detect the beginning of ischemic events (e.g., slowing of flow) or brain shift, thus allowing a medical team to intervene before an operative procedure causes a shift in brain tissue that may endanger the patient during a procedure (e.g., implanting DBS electrodes), or before a procedure causes an ischemic event within the brain.

Although the invention has been described in detail for the purpose of illustration based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An ultrasound-enabled fiducial comprising:
a proximal end configured for attachment to a patient and to bypass an outer table of the patient's skull;
a distal end; and
an ultrasound transducer.

2. The ultrasound-enabled fiducial of claim 1, wherein the proximal end comprises a screw region.

3. The ultrasound-enabled fiducial of claim 2, wherein the fiducial is self-tapping and/or self-drilling.

4. The ultrasound-enabled fiducial of claim 1, wherein the fiducial is radiopaque and MRI compatible.

5. The ultrasound-enabled fiducial of claim 1, wherein the fiducial is made from one or more of titanium, titanium alloys, cobalt chrome, and/or stainless steel.

6. The ultrasound-enabled fiducial of claim 1, wherein the fiducial is made of a polymeric material.

7. The ultrasound-enabled fiducial of claim 1, wherein the fiducial comprises a hollow shaft between the proximal end and the distal end.

8. The ultrasound-enabled fiducial of claim 7, wherein the hollow shaft is at least partially filled with an ultrasound gel.

9. A system comprising:
an ultrasound-enabled fiducial comprising:
a proximal end configured for attachment to a patient and to bypass an outer table of the patient's skull;
a distal end; and
an ultrasound transducer; and
one or more processors in communication with the transducer, the one or more processors programmed or configured to:
transmit, by a signal generator, a first electrical signal to the transducer;
receive first ultrasound data from the transducer; and
convert the first ultrasound data to a first ultrasound image.

10. The system of claim 9, wherein the one or more processors are further programmed or configured to:
store, in a memory, the first ultrasound data and/or the first ultrasound image;
transmit, by a signal generator, a second electrical signal to the transducer;
receive second ultrasound data from the transducer; and
convert the second ultrasound data to a second ultrasound image.

11. The system of claim 10, wherein the one or more processors are further programmed or configured to:
identify, based on the first ultrasound data, one or more anatomical landmarks;
identify, based on the second ultrasound data, the one or more anatomical landmarks; and
calculate, between the first ultrasound data and the second ultrasound data, a difference in location of the one or more anatomical landmarks.

12. The system of claim 10, wherein the one or more processors are further programmed or configured to:
identify, based on the first ultrasound image, one or more anatomical landmarks;
identify, based on the second ultrasound image, the one or more anatomical landmarks; and
calculate, between the first ultrasound image and the second ultrasound image, a difference in location of the one or more anatomical landmarks.

13. The system of claim 12, wherein the one or more processors are further programmed or configured to:
in response to calculating the difference in location and based on a predetermined threshold, provide a visual, audible, and/or tactile alert if the difference exceeds the predetermined threshold.

14. A method of obtaining an intracranial ultrasound image, comprising:
attaching to a patient's skull an ultrasound-enabled fiducial comprising:
a proximal end configured for attachment to a patient and to bypass an outer table of the patient's skull;
a distal end; and
an ultrasound transducer; and
generating an ultrasound image.

15. The method of claim 14, wherein the step of generating an image comprises:
providing an electrical signal to the transducer;
converting, with the transducer, the electrical signal to sound waves;
receiving, with the transducer, return sound waves;
converting, with the transducer, the return sound waves to ultrasound data;
receiving, with one or more processors and from the transducer, the ultrasound data; and
converting, with one or more processors, the ultrasound data to an ultrasound image.

16. The method of claim 14, wherein in the ultrasound-enabled fiducial comprises a bone anchor, and wherein the bone anchor is inserted into the patient's skull such that the ultrasound transducer is located interiorly of and bypasses the outer table of the patient's skull, or can transmit soundwaves through the fiducial that bypasses the outer table.

17. The method of claim 14, wherein the method further comprises the steps of:
identifying, with one or more processors, one or more anatomical landmarks in a first ultrasound image;
identifying, with one or more processors, one or more anatomical landmarks in a second ultrasound image; and
calculating, between the first ultrasound image and the second ultrasound image, a difference in location of the one or more anatomical landmarks.

18. The method of claim 17 further comprising the step of:
in response to calculating the difference in location and based on a predetermined threshold, providing a visual, audible, and/or tactile alert if the difference exceeds the predetermined threshold.

19. The method of claim 15, wherein the ultrasound data is Doppler ultrasound data and the ultrasound image is a Doppler ultrasound image, and wherein the method further comprises the step of:
introducing a contrast agent into the patient's circulation.

20. A method of detecting changes in elasticity of a patient's brain tissue, comprising:
attaching to a patient's skull an ultrasound-enabled fiducial comprising:
a proximal end configured for attachment to a patient and to bypass an outer table of the patient's skull;
a distal end; and
an ultrasound transducer;
providing an electrical signal to the transducer;
converting, with the transducer, the electrical signal to sound waves;
receiving, with the transducer, reflected sound waves;
converting, with the transducer, the reflected sound waves to first ultrasound data;
receiving, with one or more processors and from the transducer, the first ultrasound data;
converting, with one or more processors, the first ultrasound data to first elasticity data;
providing an electrical signal to the transducer;
converting, with the transducer, the electrical signal to sound waves;
receiving, with the transducer, reflected sound waves;
converting, with the transducer, the reflected sound waves to second ultrasound data;
receiving, with one or more processors and from the transducer, the second ultrasound data;
converting, with one or more processors, the second ultrasound data to second elasticity data;
calculating, with one or more processors, a difference in elasticity in the brain tissue based on the first elasticity data and the second elasticity data;
determining, with one or more processors and based on a predetermined threshold, whether the difference in elasticity exceeds the predetermined threshold; and
providing a visual, audible, and/or tactile alert if the difference exceeds the predetermined threshold.
